(19) United States
(12) Patent Application Publication
(10) Pub. No.: US 2018/0141043 A1
(43) Pub. Date: May 24, 2018

(54) Sample Collection Device and Methods for Nucleic Acid Preservation

(71) Applicants: John Alexander Malcolmson, Beverly, MA (US); Dominic Paratore, Beverly, MA (US)

(72) Inventors: John Alexander Malcolmson, Beverly, MA (US); Dominic Paratore, Beverly, MA (US)

(21) Appl. No.: 15/875,363

(22) Filed: Jan. 19, 2018

Related U.S. Application Data
(63) Continuation-in-part of application No. 15/615,761, filed on Jun. 6, 2017.
(60) Provisional application No. 62/345,921, filed on Jun. 6, 2016.

(51) Int. Cl. B01L 3/00; C12Q 1/6806; A61B 10/00

(52) U.S. Cl. CPC B01L 3/5029; B01L 3/545; A61B 10/0051; C12Q 1/6806

(57) ABSTRACT

The present application generally relates to methods and compositions for biological sample collection, including a sample collection device directed to the use of a flammable solvent-free based composition for preservation, stabilization, and transportation of nucleic acids from biological samples for long-term room temperature storage.

### CROSS-REFERENCE

This application is a continuation-in-part of U.S. application Ser. No. 15/615,761 filed Jun. 6, 2017, which claims priority to U.S. provisional application Ser. No. 62/345,921, filed Jun. 3, 2016, the contents of which are incorporated herein by reference in their entirety.

### BACKGROUND OF THE INVENTION

The present application generally relates to methods and compositions for biological sample collection and preservation, including sample collection devices involving the use of flammable solvent-free compositions for preservation, stabilization, and transportation of nucleic acids from biological samples for short-term or long-term storage at room temperature or other temperatures above or below room temperature. Embodiments described herein feature a device for collecting biological samples such as saliva containing nucleic acids, a preservative composition affixed directly to the device or to a carrier substrate and placed within the device, and a liquid preservative composition contained within the device such that the biological samples come into contact with flammable solvent-free preservative compositions.

Sample collection and preservation of biological samples, especially samples needed for collection of nucleic acids, generally exist in laboratory settings where any combination of preservation methods and storage solutions can be employed with limited practical obstacles. However, these compositions and methods are limited in point-of-care settings where the devices with compositions for preservation are shipped to end-users who then must collect and then ship the biological samples for testing.

Biological samples may be utilized to test patients for a variety of medical conditions, genetic tendencies or propensities or measure in research subjects a variety of experimental variables in a non-invasive or minimally-invasive manner However, existing solutions for the collection and long-term storage of biological samples suffer from several disadvantages, especially those that involve flammable solvent-based preservatives. For the collection and storage of saliva samples for example, existing collection devices generally use preservative compounds in liquid forms that comprise flammable solvents such as ethanol. Flammable solvents such as ethanol complicate the manufacturing process for the insertion of the liquid in a safe and practical way, and introduce additional risks when shipping, transporting or storing existing collection devices.

There is an unmet need for a user-friendly sample collection device that allows even inexperienced users of all ages to easily and reliably collect, store and transport biological samples for DNA analysis, such as saliva, in over-the-counter or research settings while avoiding the risks and disadvantages of flammable solvent-based sample preservatives. There is also an unmet need for improved preservation of nucleic acids relative to transportation and storage prior to its extraction in order to assure high quality nucleic acids. Furthermore, it would be desirable to have a device that is safe, compact and easy to use in over-the-counter or home-based applications and that is not rated as flammable, as is typical of devices that include flammable solvents such as ethanol in solvent-based preservatives. Therefore, there currently exists a need for a device and associated method that is easy to use and uses dry and/or liquid preservative chemistry to avoid the disadvantages of flammable solvent-based chemistry preservatives.

### SUMMARY OF THE INVENTION

Described herein are methods and compositions for biological sample collection, including a sample collection device directed to the use of a flammable solvent-free composition for preservation, stabilization, and transportation of nucleic acids from biological samples for short-term or long-term storage at various temperatures.

A first aspect of the subject matter described herein provides a device for preserving nucleic acids from a biological sample in a subject comprising: a main body forming a central portion of the device and having a sample collection element rigidly fixed thereto and extending coaxially therefrom; a first container removably engaged with the main body to form a first sealed cavity that contains the sample collection element, the first container also being at least substantially coaxial with the main body and forming a first end of the device; and, a second container removably engaged with the main body to form a second sealed cavity, the second container also being at least substantially coaxial with the main body, forming a second end of the device, and having a partition disposed within the second sealed cavity and traversing the entire cross-sectional area of the second container to form a first compartment and a second compartment within the second sealed cavity; wherein the first compartment is proximal to the main body comparative to the second compartment and the second compartment is distal to the main body comparative to the first compartment, wherein the first compartment contains a first preservative that is operatively coupled to the interior surface of the second container, wherein the second compartment contains a quantity of second preservative, and wherein the partition prohibits the quantity of the second preservative from entering the first compartment.

In an embodiment, the present application provides a device for preserving nucleic acids from a biological sample in a subject wherein the biological sample is saliva.

In another embodiment, the device for preserving nucleic acids from a biological sample in a subject comprises a sample collection element, wherein the element is a brush.

In an embodiment, the interior surface of the second container of the device is a perfluorinated polymer.

In another embodiment, the first preservative of the device comprises a proteolytic enzyme selected from the group consisting of proteinases, proteases, subtilisins and subtilases. In a further embodiment, the proteolytic enzyme is proteinase K.

In another embodiment, the second preservative of the device comprises and anionic surfactant. In a further embodiment, the anionic surfactant is selected from the group consisting of alkyl sulfates, alkyl ether sulfates, olefin sulfonates, alkyl sulfoacetates, alkyl ether sulfoacetates, alkyl sulfosuccinates, alkyl ether sulfosuccinates, and mixtures thereof. In another embodiment, the anionic surfactant is lithium dodecyl sulfate.

In an embodiment, the present application provides a device for preserving nucleic acids from a biological sample in a subject further comprising a barcode for sample identification. In another embodiment, the barcode for sample identification is affixed to the device for preserving nucleic acids from a biological sample in a subject.

In an embodiment, the present application provides method of preserving nucleic acids using a device described herein from a biological sample in a subject comprising: collecting the sample from the subject with a sample collection element; introducing the sample collection element into the second container; contacting the sample with the first preservative to form a liquid sample; contacting the liquid sample with a second preservative; and, sealing the second container thereby forming a preserved biological sample for storage at room temperature.

In another aspect of the invention, the present application provides a method for processing nucleic acids from a biological sample for chemical or biochemical analysis, the method comprising: providing a device comprising: (i) a main body forming a central portion of the device and having a sample collection element rigidly fixed thereto and extending coaxially therefrom; (ii) a first container removably engaged with the main body to form a first sealed cavity that contains the sample collection element, the first container also being at least substantially coaxial with the main body and forming a first end of the device; and, (iii) a second container removably engaged with the main body to form a second sealed cavity, the second container also being at least substantially coaxial with the main body, forming a second end of the device, and having a partition disposed within the second sealed cavity and traversing the entire cross-sectional area of the second container to form a first compartment and a second compartment within the second sealed cavity; wherein the first compartment is proximal to the main body comparative to the second compartment and the second compartment is distal to the main body comparative to the first compartment, wherein the first compartment contains a first preservative that is operatively coupled to the interior surface of the second container, wherein the second compartment contains a quantity of a second preservative, and wherein the partition prohibits the quantity of the second preservative from entering the first compartment; extracting nucleic acids from the second container; and, performing chemical or biochemical analysis of the nucleic acids.

In an embodiment, the present application provides a method for processing nucleic acids from a biological sample in a subject wherein the biological sample is saliva.

In another embodiment, the method for processing nucleic acids from a biological sample in a subject comprises a sample collection element, wherein the element is a brush.

In an embodiment, the interior surface of the second container of the method is a perfluorinated polymer.

In another embodiment, the first preservative of the method comprises a proteolytic enzyme selected from the group consisting of proteinases, proteases, subtilisins and subtilases. In a further embodiment, the proteolytic enzyme is proteinase K.

In another embodiment, the second preservative of the device is an anionic surfactant. In a further embodiment, the anionic surfactant is selected from the group consisting of alkyl sulfates, alkyl ether sulfates, olefin sulfonates, alkyl sulfoacetates, alkyl ether sulfoacetates, alkyl sulfosuccinates, alkyl ether sulfosuccinates, and mixtures thereof. In another embodiment, the the anionic surfactant is lithium dodecyl sulfate.

In an embodiment, the present application provides a method for processing nucleic acids from a biological sample in a subject further comprising a barcode for sample identification. In another embodiment, the barcode for sample identification.

Accordingly, it is an object of the invention not to encompass within the invention any previously known product, process of making the product, or method of using the product such that Applicants reserve the right and hereby disclose a disclaimer of any previously known product, process, or method.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description, given by way of example, but not intended to limit the disclosure solely to the specific embodiments described, may best be understood in conjunction with the accompanying drawings.

FIG. 1 illustrates a perspective view of a sample collection device according to some embodiments of the disclosure.

FIG. 2 illustrates an exploded perspective view of the sample collection device shown in FIG. 1 according to some embodiments of the disclosure.

FIG. 3 illustrates a perspective view of the sample collection device shown in FIG. 1 with one container removed and a sample collection element exposed according to some embodiments of the disclosure.

FIG. 4 illustrates a perspective view of the sample collection element partially inserted into a second container of the sample collection device according to some embodiments of the disclosure.

FIG. 5 illustrates a cross-sectional view of a main body of the sample collection device according to some embodiments of the disclosure.

FIG. 6 illustrates a perspective view of a sample collection device having an elongated handle according to some embodiments of the disclosure.

FIG. 7 illustrates a series of views showing an embodiment of the device in operation, where each step is numbered sequentially, according to embodiments of the disclosure.

### DETAILED DESCRIPTION

The present application generally relates to methods and compositions for biological sample collection, including a sample collection device directed to the preservation and stabilization of nucleic acids from biological samples for long-term storage.

The application provides a composition for use in a biological sample collection device with a flammable solvent-free preservative that affords practical manufacturing, transportation, and end-user advantages. Moreover, Applicant has discovered a composition which, when combined with the biological sample, provides increased resistance to sample degradation under dry, room-temperature conditions for periods of three years or greater.

Depending upon the application (e.g., feces collection, saliva collection, etc.), different embodiments of the sample collection device may contain various preservatives, lysing beads and swab treatments. The preservatives may cease metabolic activity, lyse cells, and preserves the nucleic acids for later extraction. Further, the preservatives prevent any unwanted growth of microbes in the sample collection device during shipment or storage.

Certain specific details are set forth in order to provide a thorough understanding of various embodiments described herein. However, one skilled in the art will understand that the subject matter of the present disclosure may be practiced without these details. Unless the context requires otherwise, throughout the present specification and claims, the word “comprise” and variations thereof, such as, “comprises” and “comprising” are to be construed in an open, inclusive sense (i.e., as “including, but not limited to”).

Reference throughout this specification to “one embodiment” or “an embodiment” means that a particular feature, structure or characteristic described in connection with the embodiment is included in at least one embodiment described herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

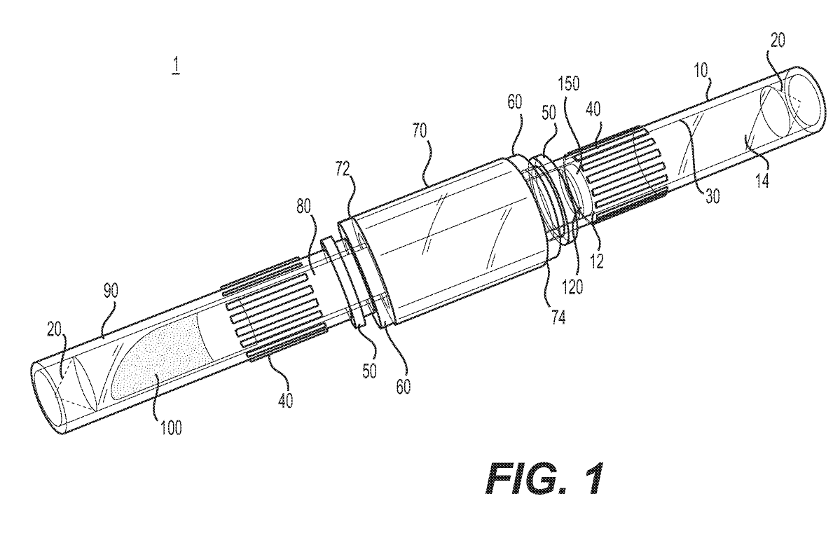

Referring to FIG. 1, the illustration provides an embodiment of a sample collection device 1 in a closed position where a main body (70) is removably engaged with a first container (90) and removably engaged with a second container (10). The first container (90) and second container (10) may be substantially rigid, for example, manufactured from a plastic material. Main body (70) may include a sample collection element comprising a shaft (80) having an end portion covered with absorbent material (100). In some embodiments the absorbent material may be flock. In some embodiments the shaft (80) may be comprised of multiple elements and include a swab or a cotton swab portion or element removably or permanently attached thereto. The sample collection element, specifically the end of the element, having the absorbent material (100) should enable the end-user to obtain the biological sample, such as saliva, from a surface, such as the inside of a buccal cavity, in a minimally-invasive manner.

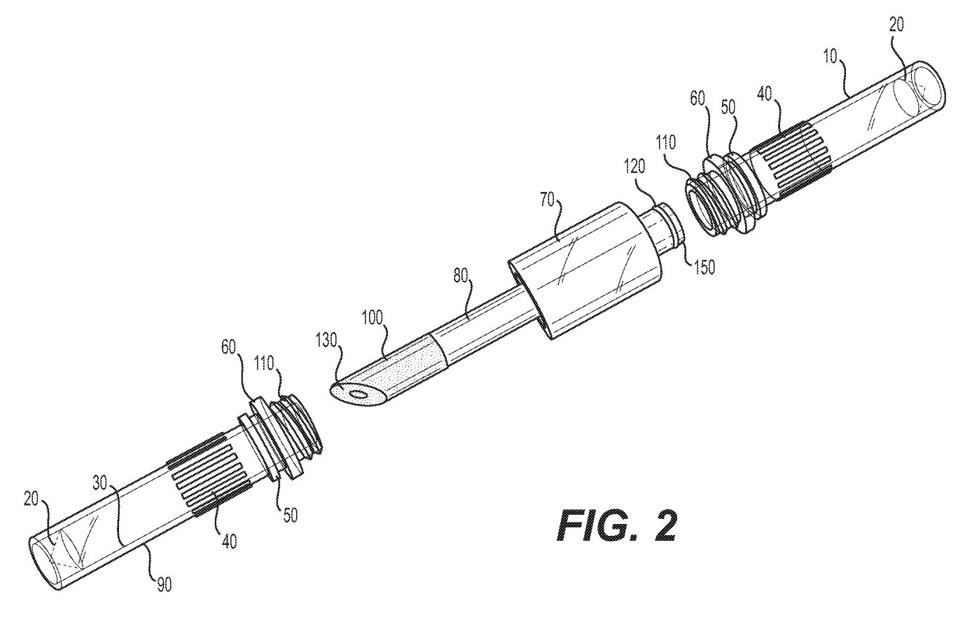

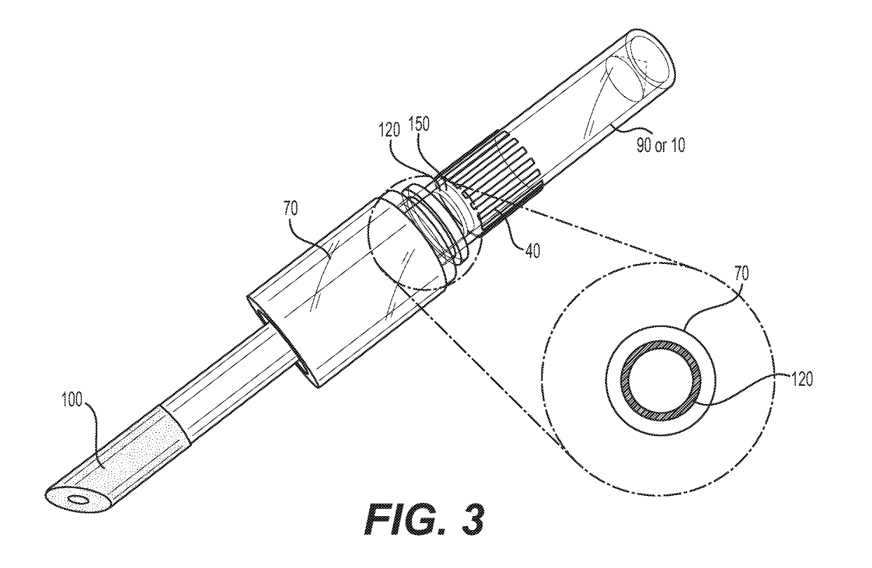

As illustrated in FIGS. 1 and 2, main body (70) may be configured with a first end (72) and a second end (74). First end (72) may be removably engaged with a first container (90) by way of threaded engagement, such as screw threads or plastic threads (110) as depicted in FIG. 2 on first container (90). Second end (74) of main body (70) may be removably engaged with a second container (10), similarly by way of threaded engagement, such as screw threads or plastic threads (110) as depicted in FIG. 2 on second container (10). It will be understood that within the first end (72) and within the second end (74) of main body (70) there are engaging threads to engage with each of the first container (90) and second container (10), respectively, as shown for example in FIG. 3. First container (90) and second container (10) may include in some embodiments a ridge (60) that provides for mating engagement and a tight fit of each container against main body (70).

Embodiments of main body (70) may include at the second end (74) an extension (150) having a groove (140) and a seal (120), such as for example, an O-ring or gasket provided therein, as shown in FIGS. 2 and 3. When main body (70) and second container (10) are threadably engaged, as shown in FIG. 1, the seal (120) provides for a first compartment (12) and a second compartment (14) within second container (10). In an embodiment of the invention, the seal (120) may be an elastomer, butadiene rubber, butyl rubber, perfluoroelastomer, silicone rubber, polytetrafluoroethylene, polyisoprene, nitrile rubber, or styrene-butadiene rubber.

In an embodiment, the second container (10) may have a conically-shaped end (20) as shown in FIGS. 1 and 2. Conically-shaped end (20) may be situated in the second compartment (14).

As illustrated in FIGS. 1 and 2, second container (10) also has an interior surface (30). In an embodiment, interior surface (30) may include a perfluorinated polymer. In an embodiment, the perfluorinated polymer is fluorinated polyethylene.

Referring to FIGS. 1 and 2, first end (72) of main body (70) is illustrated with shaft (80) having absorbent material (100) extending therefrom. Shaft (80) may be rigidly (e.g., permanently) affixed or removably affixed to main body (70). In an embodiment, shaft (80) may be configured with a slanted end as exemplified in FIGS. 1 and 2. In an embodiment, the end of shaft (80) may include comprise an indentation (130).

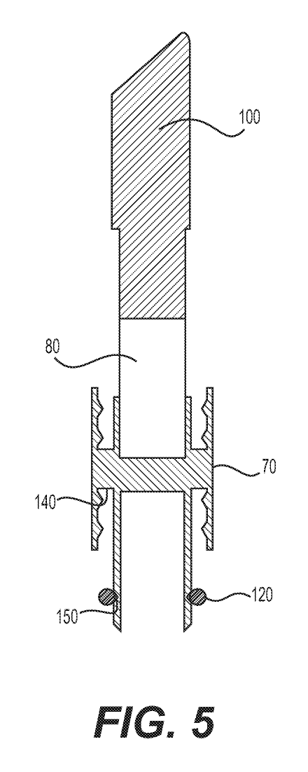

Referring to FIGS. 1 and 2, an end-user may receive the sample collection device 1 in the closed position as shown in FIG. 1. It is contemplated that the end-user will unscrew the first container (90) from the main body (70) exposing the sample collection element comprised of shaft (80) with absorbent material (100), wherein the end-user may use the sample collection element to collect saliva in the buccal cavity. As shown in FIG. 5, it is contemplated that the sample collection element is connected to both the double-ended cap and the first container (90) in order to provide a longer protruding sample collection element. In an embodiment, the exterior surface of each container includes vertical ridges (40) for gripping.

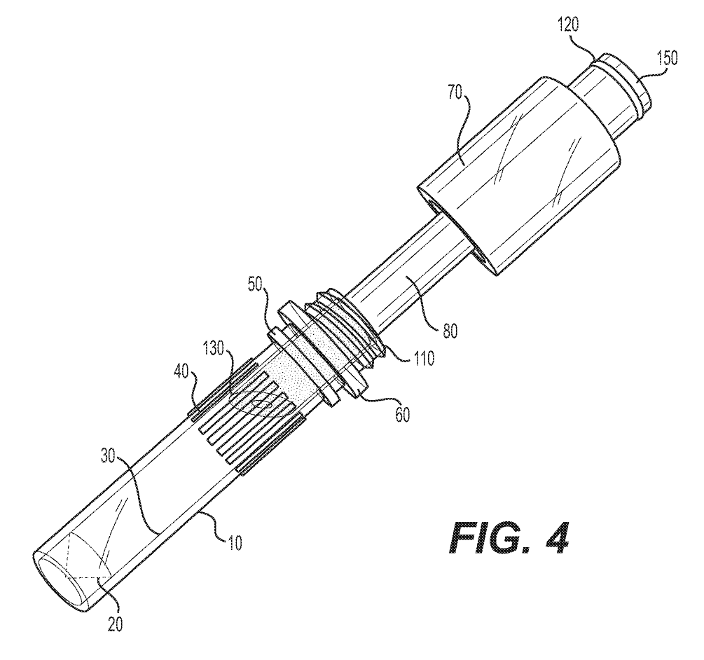

Referring to FIG. 4, once the biological sample is collected on to the absorbent material (100) of the sample collection element, the end-user will remove (e.g., unthread) the second container (10) from the main body (70) (if not already removed) and thereafter insert the sample collection element into the second container (10), for example, by threadably engaging the first end (72) of the main body (70) with the threads (110) of the second container (10), as shown in FIG. 4. During insertion of sample collection element into second container (10), the sample collection element, and in particular absorbent material (100) will first scrape against or otherwise contact with the interior surface (30) of second container (10) within the first compartment (12) and then, subsequently, the second compartment (14). In an embodiment, the interior surface (30) of the first compartment (12) of the second container (10) comprises a first preservative. In another embodiment, the first preservative is affixed to a substrate which is operably connected to the interior surface (30). In a further embodiment, the substrate may be, but not limited to, paper. In another embodiment, the substrate may be chromatography paper.

In an embodiment, the second compartment (14) of second container (10) comprises second preservative. When second container (10) is threadably engaged with the second end (74) of main body (70), such that extension (150) protrudes into second container (10), seal (120) of extension (150) is tightly pressed against or otherwise in contact with the interior surface (30) of second container (10) such that first compartment (12) and second compartment (14) are wholly sealed from each other. As a result, the second preservative contained within second compartment (14) does not and cannot enter first compartment (12) so as to contact the first preservative contained on the interior surface (30) of second container (10) that is located and isolated within first compartment (12).

FIG. 5 shows a cross-sectional view of an embodiment of the main body (70) having a sample collection element, a first end (72), a second end (74) and an extension (150).

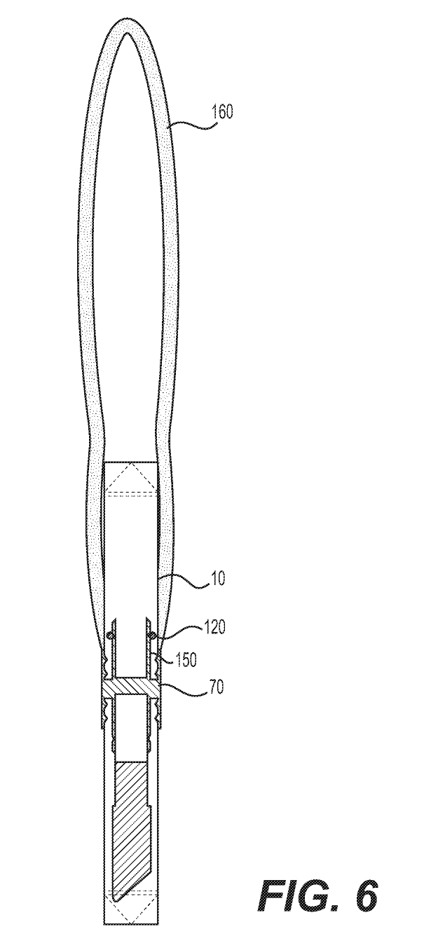

FIG. 6 shows an embodiment of a sample collection device according to the subject matter described herein having an extended handle (160) which may be used in instances of sample collection from large mammals, such as a cow or horse. The handle is removably engaged with the first container (90), for example by threaded engagement, snap engagement or a press fit.

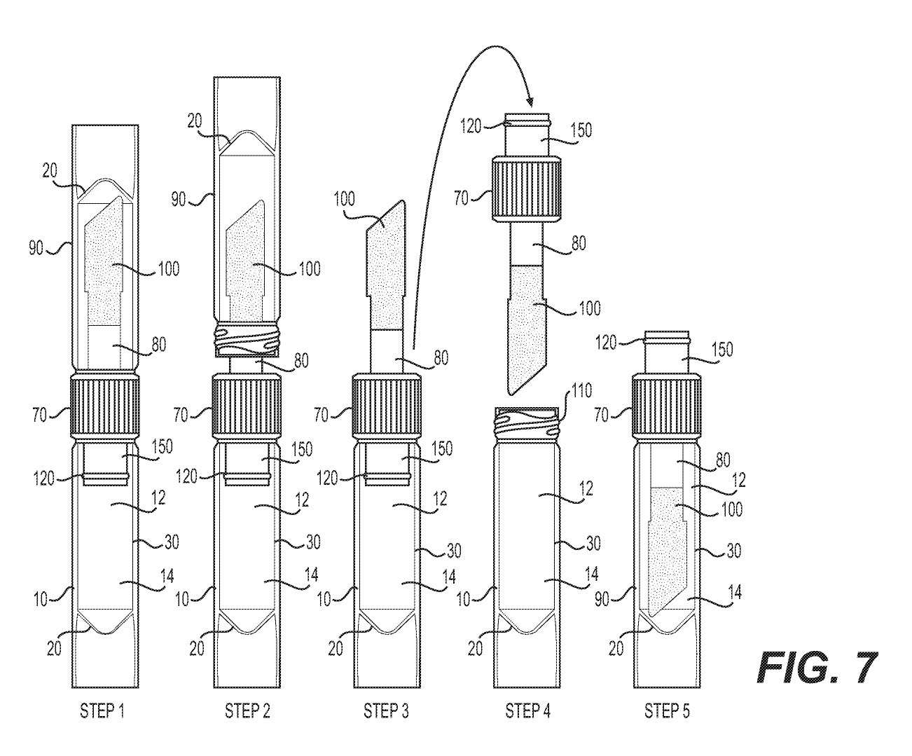

FIG. 7 shows a series of views showing an embodiment of the device in operation. Step 1 shows the device after it is removed from any packaging and is ready for use. Step 2 shows the device with the first container (90) unscrewed or detached from the main body (70) to expose the shaft (80), including absorbent material (100). Step 3 shows the shaft (80) and absorbent material (100) ready for use, where the main body (70) and either container (90) or (10) as shown in FIG. 3 can be grasped as the handle for ease of manipulation. Step 4 shows the shaft (80), absorbent material (100) and main body (70) inverted such that the sample containing element can come into contact with the first preservative and be submerged in the second preservative by way of insertion into the second container (10). Once submerged, main body (70) can be tightened to seal to second container (10). Step 5 shows the device completely closed. First container (90) may either be discarded or it can be reattached to the main body to eliminate any sampling site waste. The device is then ready for transport.

As used herein, and unless noted to the contrary, the following terms and phrases have the meaning noted below.

The term “biological sample”, as used herein, refers to a specimen of matter from an animal, such as saliva, sputum, serum, plasma, blood, urine, mucus, semen, feces, products of lactation or menstruation, tears, or lymph.

The term “sample collection element”, as used herein, refers to a brush, a swab, a damp swab, or a flock.

The term “proteolytic enzyme”, as used herein, refers to proteinases, proteases, subtilisins, and subtilases. As used herein, proteolytic enzymes may include, but is not limited to, proteinase K, endoproteinase trypsin, chymotrypsin, endoproteinase Asp-N, endoproteinase Arg-C, endoproteinase Glu-C, endoproteinase Lys-C, pepsin, thermolysin, elastase, papain, cloastripain, exopeptidase, carboxypeptidase A, carboxypeptidase B, carboxypeptidase P, carboxypeptidase Y, cathepsin C, acylamino-acid-releaseing enzyme, pyroglutamate, subtilisin, or aminopeptidase.

The use of a surfactant in pharmaceutical compositions is well-known to the skilled person. For convenience, reference is made to Remington: The Science and Practice of Pharmacy, 19th edition, 1995.

Anionic surfactants may be selected from the group of: chenodeoxycholic acid, chenodeoxycholic acid sodium salt, cholic acid, dehydrocholic acid, deoxycholic acid, deoxycholic acid methyl ester, digitonin, digitoxigenin, N,N-dimethyldodecylamine N-oxide, docusate sodium, glycochenodeoxycholic acid sodium, glycocholic acid hydrate, glycodeoxycholic acid monohydrate, glycodeoxycholic acid sodium salt, glycolithocholic acid 3-sulfate disodium salt, glycolithocholic acid ethyl ester, N-lauroylsarcosine sodium salt, N-lauroylsarcosine sodium salt, N-lauroylsarcosine, N-lauroylsarcosine, lithium dodecyl sulfate, lugol, 1-octanesulfonic acid sodium salt, sodium 1-butanesulfonate, sodium 1-decanesulfonate, sodium 1-dodecanesulfonate, sodium 1-heptanesulfonate, sodium 1-nonanesulfonate, sodium 1-propanesulfonate monohydrate, sodium 2-bromoethanesulfonate, sodium cholate hydrate, ox or sheep bile, sodium cholate hydrate, sodium choleate, sodium deoxycholate, sodium dodecyl sulfate, sodium dodecyl sulfate, sodium hexanesulfonate, sodium octyl sulfate, sodium pentanesulfonate, sodium taurocholate, taurochenodeoxycholic acid sodium salt, taurodeoxycholic acid sodium salt monohydrate, taurolithocholic acid 3-sulfate disodium salt, tauroursodeoxycholic acid sodium salt, Trizma® dodecyl sulfate, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), dodecylphosphocholine (FOS-choline-12), decylphosphocholine (FOS-choline-10), nonylphosphocholine (FOS-choline-9), dipalmitoyl phosphatidic acid, sodium caprylate, and/or ursodeoxycholic acid.

Cationic surfactants may be selected from the group of: alkyltrimethylammonium bromide, benzalkonium chloride, benzyldimethylhexadecylammonium chloride, benzyldimethyltetradecylammonium chloride, benzyltrimethylammonium tetrachloroiodate, dimethyldioctadecylammonium bromide, dodecylethyldimethylammonium bromide, dodecyltrimethylammonium bromide, dodecyltrimethylammonium bromide, ethylhexadecyldimethylammonium bromide, hexadecyltrimethylammonium bromide, hexadecyltrimethylammonium chloride, polyoxyethylene-(10)-N-tallow-1,3-diaminopropane, thonzonium bromide, and/or trimethyl(tetradecyl)ammonium bromide.

In some embodiments, a formulation of the second preservative contained in second container (10) may include protease inhibitors such as EDTA (ethylenediamine tetraacetic acid) and benzamidine HCl, but other commercially available protease inhibitors may also be used. The use of a protease inhibitor is particular useful in pharmaceutical compositions comprising zymogens of proteases in order to inhibit autocatalysis.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces.

An “alkyl” group refers to an aliphatic hydrocarbon group. The alkyl groups may or may not include units of unsaturation. The alkyl moiety may be a “saturated alkyl” group, which means that it does not contain any units of unsaturation (i.e. a carbon-carbon double bond or a carbon-carbon triple bond). The alkyl group may also be an “unsaturated alkyl” moiety, which means that it contains at least one unit of unsaturation. The alkyl moiety, whether saturated or unsaturated, may be branched, straight chain, or cyclic. By way of example only, an alkyl chain is selected from the group consisting of methyl, ethyl, n-propyl, iso-propyl, n-butyl, iso-butyl, sec-butyl, t-butyl, n-pentyl, iso-pentyl, neo-pentyl, hexyl, propen-3-yl (allyl), cyclopropylmethyl, cyclobutylmethyl, cyclopentylmethyl, cyclohexylmethyl. Alkyl groups can be substituted or unsubstituted. Depending on the structure, an alkyl group can be a monoradical or a diradical (i.e., an alkylene group).

A “patient,” “subject,” or “end-user” is a vertebrate, preferably a mammal, and often a human. Mammals include, but are not limited to, humans, non-human primates, experimental model animals (e.g., mice and rats), agriculturally important animals, and pets.

### EXAMPLES

The following examples are offered for purposes of illustration, and are not intended to limit the scope of the claims provided herein. All literature citations in these examples and throughout this specification are incorporated herein by references for all legal purposes to be served thereby.

In some aspect of the subject matter described herein, the application provides a composition for use in a device for collection and preservation of biological samples. A sample collection element having a brush or swab, or damp swab (e.g., flock) may be used to collect a biological sample, such as saliva, from an orifice, such as the buccal cavity. Some devices in the field have used collection vials where the end-user must spit into the vial. These devices thus require a large amount of saliva, which is difficult for some subjects to provide, especially young subjects. Applicant found that the use of a sample collection element such as a swab provides more discrete action and broader patient population application.

In an embodiment of a procedure for operating the device, the end-user will receive the device in the closed position as illustrated in FIG. 1, wherein main body (70) is removably connected to a first container (90) and a second container (10). The end-user may either remove the second-container from the main body prior to collection of the biological sample or after collection. The end-user will obtain a biological sample using the sample collection element, wherein the biological sample will either adhere or absorb to the end of the element (100). Second container (10) may be engaged to main body (70) as illustrated in FIG. 3 to provide a handle for sample collection. In an embodiment, a handle may be used to provide a longer handle for sample collection as illustrated in FIG. 6. In an aspect of the operating procedure, an extension (150) having a seal (120) projects into second container (10) to form a first compartment (12) and a second compartment (14) that are sealed off from one another so as to prohibit any fluid communication therebetween. When second container (10) and main body (70) are separated, for example by unthreading one from the other, the first compartment (12) and second compartment (14) are no longer sealed off and fluid communication therebetween is permitted. In a further aspect, the sample collection element is introduced into the second container (10), wherein the biological sample that has been collected on the absorbent material (100) of the shaft (80) of the sample collection element is put into contacted with a first preservative located on the interior surface (30) of the first compartment (12) of second container (10). The second container of the sample collection device provides a tight fit around shaft (80) such that the sample collection element scours the interior surface of the first compartment (12) where the absorbent material (100) is coated with Ingredient H, e.g, proteinase K. The sample collection continues to move further into the container into the second compartment (14) wherein the absorbent material is submerged in the second compartment.

In an embodiment, the first container and second container comprise a polyfluorinated polymer on the interior surface. In an aspect of the operating procedure, the fluorinated polymer of the interior surface (30) allows the higher adhesion of the preservatives, such as proteinase K.

In an embodiment the dry preservative comprises a blend of reagents and enzymes that allow for complete lysis and stabilization of the nucleic acids DNA and RNA, ease of transportation and long-term storage of samples under ambient conditions prior to chemical or biological analysis. The ingredients of the preservative may comprise the following: Ingredient A—enzyme denaturation, including nucleases, DNase enzymes (deoxyribonuclease), and the like; Ingredient B—metal chelating cofactors required by DNase enzymes; Ingredient C—enzyme denaturing agents that helps reduce viscosity of saliva along with other supporting chemicals; Ingredient D—hydration agent; Ingredient E—pH buffer; Ingredient F—DNA and RNA precipitation agent; Ingredient G—a chemical which modifies sample viscosity and surface tension; Ingredient H—sample nuclease digestion agent.

In an embodiment, the ingredients are combined to form two separate preservatives. In another embodiment, the first preservative comprising ingredients A through F may also comprise ingredient G. In an embodiment, the second preservative comprises ingredient H and may also comprise ingredient F and/or G.

Ingredient A may be a substance such as lithium dodecyl sulfate or similar. Applicant found that use of ingredient A, such as lithium dodecyl sulfate (LDS), prevented the composition from precipitating at low temperatures once the liquid sample was formed. Applicant found that other substances generally used in the art precipitate out of solution at temperatures below 17° C. and then unable to go back into solution unless the pH ranges from 10-11.

Ingredient B may be a substance such as 1,2-cyclohexanediaminetetraacetic acid (CDTA), ethylenediaminetetracetic acid (EDTA) or similar.

Ingredient C may be a substance such as urea or similar. Ingredient D may be a substance such as 1,2-propanediol or similar. Ingredient E may include Tris (around 100 mM concentration) and NaCl (around 0.3 M final concentration). In an alternate embodiment, NaOAc (sodium acetate) may be used in place of NaCl.

Ingredient F may be a substance such as polyethylene glycol 8000 or similar. Applicant found that use of Ingredient F, such as polyethylene glycol (PEG) which may be used independently in the first preservative and in the second preservative, afforded a significantly higher concentration of nucleic acids upon extraction. In an embodiment of the application, polyethylene gycol is used in range of 30-40 w/w %. With the use of 30-40% w/w of PEG, the shelf-life or preservation period afforded at least three (3) years under dry, room-temperature conditions. Moreover, the use of polyethylene glycol is not known to be used in commercial kits for the purpose of obtaining a higher nucleic acid concentration. Thus, the composition provides a low-cost, safe, and efficient chemical which synergistically provides a significantly higher amount of nucleic acid extraction and long-term storage of at least three years under ambient conditions.

Ingredient G may be a surfactant such as hexadecyltrimethylammonium chloride or similar. The use of Ingredient G (e.g., hexadecyltrimethylammonium chloride) affords, amongst several uses, an antibacterial agent. The use of Ingredient G also provides an agent for reducing the viscosity of the biological sample. It is understood by one of skill in the art, that biological samples, such as saliva, are viscous due to the mucopolysaccharides which therefore make testing of these samples difficult. Previously known techniques used in laboratory settings include centrifugation and filtration. In an aspect of the operating procedure, the nucleic acids encompassed by the saliva must become “exposed” in order for the preservatives to react with the nucleic acids. Without being bound to any one theory, viscosity reduction by Ingredient G is caused by the chemical interaction between the polyanionic mucopolysaccharides (comprising neuraminic acid and sulfated residues). For example, electrostatic interaction between hexadecyltrimethylammonium chloride and saliva mucopolysaccharides produces an insoluble aggregate. When the hydrated chloride ion is displaced by the anionic mucopolysaccharide, the quaternary ammonium complex becomes insoluble. In an embodiment of the invention, hexadecyltrimethylammonium chloride is used in approximately 0.01±0.005 M concentration.

Ingredient H may be a substance such as Proteinase K or similar. In some embodiments, Ingredient H may be located in the second container (10) in the first compartment (12). The ingredient, such as proteinase K, is kept separate from the second preservative until the sample collection element is inserted and submerged into the second compartment (14).

In an embodiment of the invention, the composition comprises sodium chloride (NaCl). The use of NaCl improves the ability of the preservative to handle elevated temperatures and improves shelf-life. Applicant found that the sample will brown (i.e., residual proteins are browning) at elevated temperatures. The addition of NaCl causes large DNA to precipitate out prior to extraction. Thus, the use of NaCl interferes with browning; provides DNA preservation; assists precipitation of large, undigested DNA; and is especially compatible with ingredient A (e.g., LDS). In an embodiment of the invention, NaCl is used in the range of approximately 0.3 to 0.5 M in solution.

The examples and embodiments described herein are for illustrative purposes only and in some embodiments, various modifications or changes are to be included within the purview of disclosure and scope of the appended claims.

What is claimed is:

1. A device for preserving nucleic acids from a biological sample in a subject comprising:
(a) a main body forming a central portion of the device and having a sample collection element rigidly fixed thereto and extending coaxially therefrom;
(b) a first container removably engaged with the main body to form a first sealed cavity that contains the sample collection element, the first container also being at least substantially coaxial with the main body and forming a first end of the device; and,
(c) a second container removably engaged with the main body to form a second sealed cavity, the second container also being at least substantially coaxial with the main body, forming a second end of the device, and having a partition disposed within the second sealed cavity and traversing the entire cross-sectional area of the second container to form a first compartment and a second compartment within the second sealed cavity;
wherein the first compartment is proximal to the main body comparative to the second compartment and the second compartment is distal to the main body comparative to the first compartment,
wherein the first compartment contains a first preservative that is operatively coupled to the interior surface of the second container,
wherein the second compartment contains a quantity of a second preservative, and
wherein the partition prohibits the quantity of the second preservative from entering the first compartment.

2. The device of claim 1, wherein the biological sample is saliva.

3. The device of claim 1, wherein the sample collection element is a brush.

4. The device of claim 1, wherein the interior surface of the second container is a perflourinated polymer.

5. The device of claim 1, wherein the first preservative comprises a proteolytic enzyme selected from the group consisting of proteinases, proteases, subtilisins and subtilases.

6. The device of claim 5, wherein the proteolytic enzyme is proteinase K.

7. The device of claim 1, wherein the second preservative comprises an anionic surfactant.

8. The device of claim 7, wherein the anionic surfactant is selected from the group consisting of alkyl sulfates, alkyl ether sulfates, olefin sulfonates, alkyl sulfoacetates, alkyl ether sulfoacetates, alkyl sulfosuccinates, alkyl ether sulfosuccinates, and mixtures thereof.

9. The device of claim 7, wherein the anionic surfactant is lithium dodecyl sulfate.

10. The device of claim 1, further comprising a barcode for sample identification.

11. A method of preserving nucleic acids using a device according to claim 1 from a biological sample in a subject comprising:
(a) collecting the sample from the subject with a sample collection element;
(b) introducing the sample collection element into the second container;
(c) contacting the sample with the first preservative to form a liquid sample;
(d) contacting the liquid sample with the second preservative; and,
(e) sealing the second container thereby forming a preserved biological sample for storage at room temperature.

12. A method for processing nucleic acids from a biological sample for chemical or biochemical analysis, the method comprising:
(a) providing a device comprising:
(i) a main body forming a central portion of the device and having a sample collection element rigidly fixed thereto and extending coaxially therefrom;
(ii) a first container removably engaged with the main body to form a first sealed cavity that contains the sample collection element, the first container also being at least substantially coaxial with the main body and forming a first end of the device; and,
(iii) a second container removably engaged with the main body to form a second sealed cavity, the second container also being at least substantially coaxial with the main body, forming a second end of the device, and having a partition disposed within the second sealed cavity and traversing the entire cross-sectional area of the second container to form a first compartment and a second compartment within the second sealed cavity;
wherein the first compartment is proximal to the main body comparative to the second compartment and the second compartment is distal to the main body comparative to the first compartment,
wherein the first compartment contains a first preservative that is operatively coupled to the interior surface of the second container,
wherein the second compartment contains a second preservative, and
wherein the partition prohibits the second preservative from entering the first compartment;
(b) extracting nucleic acids from the second container; and,
(c) performing chemical or biochemical analysis of the nucleic acids.

13. The method of claim 12, wherein the biological sample is saliva.

14. The method of claim 12, wherein the sample collection element is a brush.

15. The method of claim 12, wherein the interior surface of the second container is a perflourinated polymer.

16. The method of claim 12, wherein the first preservative comprises a proteolytic enzyme selected from the group consisting of proteinases, proteases, subtilisins and subtilases.

17. The method of claim 12, wherein the proteolytic enzyme is proteinase K.

18. The method of claim 12, wherein the second preservative comprises an anionic surfactant.

19. The method of claim 12, wherein the anionic surfactant is selected from the group consisting of alkyl sulfates, alkyl ether sulfates, olefin sulfonates, alkyl sulfoacetates, alkyl ether sulfoacetates, alkyl sulfosuccinates, alkyl ether sulfosuccinates, and mixtures thereof.

20. The method of claim 12, further comprising a barcode for sample identification.
